(19) United States
(12) Patent Application Publication
(10) Pub. No.: US 2011/0165558 A1
(43) Pub. Date: Jul. 7, 2011

(54) METHOD FOR IDENTIFYING INDIVIDUAL VIRUSES IN A SAMPLE

(76) Inventors: Jürgen Popp, Kunitz (DE); Volker Deckert, Jena (DE); Dieter Naumann, Berlin (DE); Robert Möller, Jena (DE); Dana Cialla, Jena (DE)

(21) Appl. No.: 13/062,931

(22) PCT Filed: Jul. 21, 2009
(86) PCT No.: PCT/DE2009/001031

§ 371 Date: Mar. 9, 2011

(30) Foreign Application Priority Data
Sep. 11, 2008 (DE) 10 2008 047 240.9

(51) Int. Cl. C12Q 1/70

(52) U.S. Cl. 435/5

(57) ABSTRACT

Individual viruses in any type of sample are identified quickly, unambiguously and reliably, and with the least possible preparation-related and technology-related expenditure, without necessitating immobilization using antibodies and without requiring an indication or at least a suspicion of potentially present viruses. This is accomplished by scanning the height profile of the sample, from which scanning sites suspected of containing viruses are selected, exposing those cites to monochromatic excitation light and spectroscopically analyzing the resulting Raman scattered light.

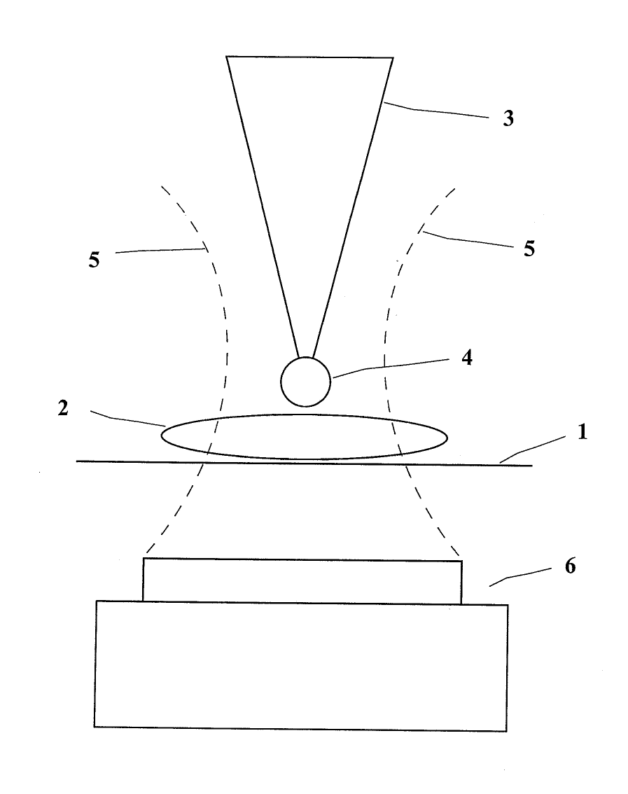

### BACKGROUND OF THE INVENTION

The invention relates to a method for identifying individual viruses in a sample quickly and reliably and with the least possible effort and expenditure.

Infection tests can be considered the oldest methods applied for the detection of virus contaminations. In these tests, bacteria and cell cultures are infected with potentially virus-containing material. After a defined time of incubation the virus infection can be shown by a visually detectable change (lytic scheme) in the cell or bacteria culture. But this detection requires a lot of time and only allows a general virus test but not the exact determination of the virus type. Moreover, a virus attack does not always result in the lysis of the infected host cells. It may be that the selected test conditions are not optimal for the viral proliferation or even inhibit it or the virus is in a so called lysogenic cycle and the DNA of the virus or phage is integrated in the DNA of the host cell so that a lytic scheme will not be developed although viruses have really attacked the cell and then that virus attack cannot be detected or determined in this way.

To overcome these disadvantages, other molecular biological methods are often used for the routine detection of viruses and bacteriophages today. The detection methods primarily applied are ELISA (e.g. S. S. Nielsen, N. Toft: Ante mortem diagnosis of paratuberculosis: A review of accuracies of ELISA, interferon-γ assay and faecal culture techniques, Veterinary Microbiology 2008, 129, 217-235) or PCR (S. Antinori, S. Calattini, E. Longhi, G. Bestetti, R. Piolini, C. Magni, G. Orlando, M. Gramiccia, V. Acquaviva, A. Foschi, S. Corvasce, C. Colomba, L. Titone, C. Parravicini, A. Cascio, M. Corbellino: Clinical Use of Polymerase Chain Reaction Performed on Peripheral Blood and Bone Marrow Samples for the Diagnosis and Monitoring of Visceral Leishmaniasis in HIV-Infected and HIV-Uninfected Patients: A Single-Center, 8-Year Experience in Italy and Review of the Literature, Clinical Infectious Diseases 2007, 44, 1602-1610; J. Peccia, M. Hernandez: Incorporating polymerase chain reaction-based identification, population characterization, and quantification of microorganisms into aerosol science: A review, Atmospheric Environment 2006, 40, 3941-3961; L. A. Benvenuti, A. Roggerio, N. V. Sambiase, A. Fiorelli, M. de Lourdes Higuchi: Polymerase chain reaction in endomyocardial biopsies for monitoring reactivation of Chagas' disease in heart transplantation—A case report and review of the literature, Cardiovascular Pathology 2005, 14, 265-268). But it is a disadvantage of these methods that the detection of individual viruses is very difficult, if not possible at all. Therefore, an incubation of bacteria and cell cultures is also used for these methods first to increase the concentration of the viruses. This cultivation requires much time and effort, too. Then, the real detection of the viruses is realized by the is detection of their DNA or RNA (PCR) or by an immunological test (ELISA).

In the PCR, the hereditary material (RNA or DNA) of the virus is reproduced and analyzed then. The hereditary material of the virus must be isolated from the sample and, if possible, be separated from cells, cellular components and other factors that can inhibit the enzymatic reaction. This sample preparation is very time and labor consuming. For the PCR, expensive reagents and specific primers must be added to the isolated hereditary material. The primers are short single-stranded DNA fragments which ensure that only certain specific parts of the viral genome, which can be used for the exact identification of the virus later, are reproduced. That means that if the PCR preparation contains the wrong primers the amplification of DNA does not occur and the viruses cannot be detected. Therefore, specific primers must be developed to detect specific viruses of different families and types. But slight changes in the hereditary material of the viruses can prevent that the primers bind to the DNA and thus the DNA does not amplify either. Viruses for which a reliable primer system does not exist cannot be identified at all by means of PCR.

The immunological detection, such as ELISA, utilizes the specific reaction between the virus particles and the antibodies. But viruses in low concentrations can only be detected after an incubation and amplification. Such methods require specific biomolecules for the virus detection, too. The immunological detection uses antibodies that are obtained from laboratory animals or cell cultures. Like the PCR process, the immunological detection also requires the selection of the correct biomolecules (PCR: primers; immunological detection: antibodies) to identify a specific virus. If antibodies do not exist or wrong antibodies are used, the virus detection will not be possible. Furthermore, the production of the antibodies requires much time and effort and an additional immobilization step is necessary in which either the viruses or the antibodies are bound to a solid substrate.

Imaging techniques are also known as methods for detecting viruses. In diagnostics, the transmission electron microscopy (TEM) is used in is combination with negative staining as an electron microscopic detection method. This method allows assigning single viruses to virus families because the fine structures of the individual virus families show sufficiently significant differences (M. Gentile, H. R. Gelderblom: Rapid viral diagnosis: role of electron microscopy, The New Microbiologica 2005, 28, (1), 1-12; P. R. Hazelton, H. R. Gelderblom: Electron microscopy for rapid diagnosis of infectious agents in emergent situations, Emerging Infectious Diseases 2003, 9, 294-303). But the transmission electron microscopy does not go beyond the exact assignment of the viruses to a virus family. Moreover, this method requires much apparatus-related and preparation-related expenditure.

Another imaging technique used is the atomic force microscopy (AFM) (Y. F. Drygin, O. A. Bordunova, M. O. Gallyamov, I. V. Yaminsky: Atomic force microscopy examination of tobacco mosaic virus and virion RNA, FEBS Letters 1998, 425, (2), 217-221). In this imaging method the virus particles that are immobilized on a solid and extremely smooth sample carrier are scanned by a very fine tip. Then, the viruses can be assigned to a specific family by analyzing the size and shape of the imaged particles. But the obtained data can be easily misinterpreted (false-positive results), in particular if the sample is contaminated with particles of other origin that have a similar size and shape and therefore cause confusion. Neither it is possibly to detect very small virus particles, if the surface of the sample carrier is too rough or too many particles are located on it.

A serious disadvantage of all imaging techniques is the fact that information about the composition of the imaged particles cannot be obtained. Thus, an assignment and determination of the particles is only realized via their shape and size so that confusion and consequently misinterpretations are particularly caused by spherical viruses.

Information about the structure of virus particles can be principally got by using spectroscopic techniques, such as the Raman spectroscopy. This vibrational spectroscopic investigation allows the assignment of the viruses to a family or type thanks to the spectra obtained. But the Raman effect on which this technique is based is very weak and thus the Raman spectroscopy method can only be used for bulk material (G. J. Thomas Jr.: Raman spectroscopy and virus research, Applied Spectroscopy 1976, 30, (5), 483-94; T. A. Turano, K. A. Hartman, G. J. Thomas Jr.: Studies of virus structure by laser-Raman spectroscopy, 3. Turnip yellow mosaic virus, Journal of Physical Chemistry 1976, 80, (11), 1157-63). That means that the viruses must be available in a high concentration and with as few contaminations as possible. The determination of individual viruses is impossible in this method.

The so called surface enhanced Raman spectroscopy (SERS) can be applied to identify poor virus concentrations. This method uses metallic nano-structures and -particles to increase the low sensitivity of Raman spectroscopy. However, this method cannot be employed in routine diagnostics because the enhancement of the Raman effect considerably varies in dependence on the nano-structures and -particles used and thus a reliable detection is not possible. Furthermore, this method does not allow the identification of individual viruses either (S. Shanmukh, L. Jones, J. Driskell, Y. Zhao, R. Dluhy, R. A. Tripp: Rapid and Sensitive Detection of Respiratory Virus Molecular Signatures Using a Silver Nanorod Array SERS Substrate, Nano Letters 2006, 6, (11), 2630-2636).

To increase the local resolution of the Raman measurement the surface enhanced Raman spectroscopy is combined with imaging techniques, e.g. AFM (atomic force microscopy) and STM (scanning tunneling microscopy). The resulting method is called tip-enhanced Raman spectroscopy (TERS) (R. M. Stöckle, Y. D. Suh, V. Deckert, R. Zenobi: Nanoscale chemical analysis by tip-enhanced Raman spectroscopy, Chemical Physics Letters 2000, 318, 131-136).

In recent years, an application for analyzing bacteria and RNA was also promoted in the field of biological issues. For the investigation of bacteria, a silver vapor coated AFM tip was placed on the bacterium. Due to the different sizes of a bacterium and of the silver vaporized probe only a very small section of the bacterium surface can be sampled. Therefore, these tests have not shown encouraging results either. The detected TERS spectra are not reproducible among each other, a fact that is indicates a mobility in the outer cell wall so that experts consider this method unsuitable for identifying bacteria (U. Neugebauer, P. Rösch, M. Schmitt, J. Popp, C. Julien, A. Rasmussen, C. Budich, V. Deckert: On the Way to Nanometer-Sized Information of the Bacterial Surface by Tip-Enhanced Raman Spectroscopy, ChemPhysChem 2006, 7, 1428-1430). In addition to this, TERS investigations were carried out for single-stranded polyC-RNA. The result shall lead to a direct sequence analysis of isolated DNA or RNA (E. Bailo, V. Deckert: Tip-Enhanced Raman Spectroscopy of Single RNA Strands: Towards a Novel Direct-Sequencing Method, Angewandte Chemie [Applied Chemistry] Int. Ed. 2008, 47, 1658-1661).

Nothing is known about the application of this method for the detection and identification of individual viruses.

Considering all methods currently applied in virus diagnostics it must be stated that a method for the reliable and unambiguous identification of individual viruses that is detection-sensitive and quick, requires low effort and can be employed even in clinic routine checks is not known at present.

### SUMMARY OF THE INVENTION

The object of the invention is to identify individual viruses in a solid, liquid or gaseous sample quickly, unambiguously and reliably, and with the least possible preparation-related and technology-related expenditure, without necessitating immobilization by using antibodies and without requiring an indication or at least a suspicion of potentially present viruses.

The method of the invention has been developed to detect individual viruses in any type (solid, liquid or gaseous) of sample and to identify the specific type of these viruses or bacteriophages without time-consuming and material-intensive preparations. Thanks to the invention it will be possible to get reliable information about the type and composition of the virus particles in a sample thus allowing the exact and unambiguous identification of these particles. This method can be universally applied to all viruses independently of the cells that are attacked by the viruses and the type of the individual virus. As the method can determine the viruses regardless of their origin it can also be used in other fields of application (e.g. for the detection of tobacco mosaic viruses in plants, for the detection of virus particles in the air, or for the detection of viruses and bacteriophages in biotechnological production). The term “virus” as used herein includes all viruses, that is, bacteriophages or “phages” as well as all other viruses. Therefore, when “viruses” or a “virus” is referred to herein without specific mention of “bacteriophages” or “phages”, it is always intended that bacteriophages or phages be included as well.

According to the invention, the height profile of a carrier surface, to which the sample to be examined is fixed, is scanned by a probe, for example by means of the AFM technique known per se. On the basis of said height profile, which is obtained by surface scanning, scanning sites that due to their surface structure are suspected of containing viruses are selected (either simultaneously with said scanning procedure or thereafter). Each of these scanning sites selected according to the height profile is exposed to monochromatic excitation light and analyzed spectroscopically with respect to the Raman scattered light resulting from the light excitation at the scanning site. The results obtained in this analysis of the Raman scattered light are compared with reference values, particularly with reference values of an electronic database, to identify the individual virus present at the scanning site.

In this method it is proposed to link, for the first time, information about the shape and size of potentially present virus particles with vibrational spectroscopic data to identify, for the first time, viruses and even individual viruses unambiguously and quickly. This aim is achieved by coupling an imaging technique (surface scanning) with the Raman spectroscopy.

Information about the number and type of viruses existing or possibly existing in the sample does not have to be necessarily available a priori but the virus structures that have been found and are to be defined at the scanning sites of the sample surface, which have been selected because of the detection of said virus structures, are analyzed and thus reliably identified by comparing their vibrational spectroscopic data with all data that are available as reference values (all detailed virus information).

Compared to the conventional methods described above, the inventive method offers many advantages: It does not require expensive molecular-biological reagents and allows an unambiguous and comparatively quick identification of viruses and even individual viruses, and the time-expensive and complex pre-cultivation and sample preparation are not necessary any longer. Thus, this invention is considerably more exact and reliable and requires less time and effort than the methods of virus detection mentioned before. Moreover, even individual viruses are not only analyzed by their shape as described but additionally by vibrational spectroscopy as recommended. Apart from data about size and structure, exact information about the chemical composition of the analyzed particles is also gathered in this method so that it allows the type-specific and unambiguous determination of these viruses for the first time.

Unlike immunological or PCR virus detection methods, the inventive method does not use molecular-biological detection reactions so that the complex sample pre-treatment and preparation of these molecular-biological processes is not necessary. Furthermore, the frequently high costs of the primers, antibodies, enzymes and other reagents required for these detection methods are saved.

Contrary to the just imaging procedures (AFM, TEM) the user can obtain very detailed information about the material composition of the scanned sample for high detection sensitivity by coupling an imaging technique with a vibrational spectroscopic method thus also allowing a very detailed structure comparison with reference data and thus an extremely precise and unambiguous virus identification and determination.

This advantage is particularly interesting for viruses that have the same or similar shape and size and cannot be clearly analyzed by topographic information alone.

According to this invention even the detection of individual viruses is possible so that a minimum concentration is not required for the reliable identification and determination and the corresponding pre-cultivations can be omitted. Only sizing by pre-treatment of the sample, for example by filtration or homogenization, is an advantageous sample preparation for the application of the invention in order to separate the large particles from the viruses and thus to purify the sample (i.e., increase the concentration of viruses in the sample) and simplify the analysis, i.e. simplify the selection of the scanning sites on the basis of the height profile of the carrier surface as well as the virus detection and determination. For this purpose, for example, a filter array with decreasing pore size is used to filter the sample. Then, the viruses in enriched form are located on a filter with the corresponding pore size and are separated from larger particles such as dust or bacteria. The sample which is filtered may be, for example a liquid or a gas. The sample may be filtered directly or, for example, a gaseous sample may first be dissolved in a liquid and the liquid solution be filtered. An example of sizing by homogenization of the sample is mechanical comminution of a solid sample.

In the following the invention is explained in more detail by the embodiments represented in the FIGURE.

### BRIEF DESCRIPTION OF THE DRAWING

The FIGURE shows the schematic arrangement for scanning the height profile of a sample, which is fixed on a carrier, by means of the AFM technique (atomic force microscopy) known per se and for analyzing the laser-excited Raman scattered light.

### DETAILED DESCRIPTION OF THE INVENTION

The sample is exposed to laser light emitted from below by a microscope objective arranged opposite to the probe. The amplified Raman signal is collected by the same microscope objective that is used for irradiating the sample and then the collected signal is led to the analysis detector of the laser microscope.

The sample to be analyzed consists of a carrier 1 with a virus 2, which is also shown in a schematic view, and is scanned in its height profile by an AMF tip 3 that is provided with a metal particle 4 at its end directed towards the sample. In this height profile scanning of the sample the prominent and suspectedly virus-containing structure of the virus 2 fixed on the carrier 1 is detected at the scanning site shown in the FIGURE. According to the invention, the process of detecting and selecting the scanning site (simultaneously with said profile scanning or after the completed height profile scanning) is combined with the analysis of the Raman scattered light of the sample that is generated at said selected site. For this purpose, a focused laser beam 5 is guided to the sample via an objective 6 of a laser microscope that is not shown in detail in the interest of clarity. Due to light excitation of the sample by the laser beam 5 scattered light is generated and the objective 6 of the laser microscope is used for detecting and analyzing it. The virus present at the scanning site of the sample is identified by comparing the analysis data of said scattered light with reference values, particularly of a database that is not shown in the FIGURE.

### Embodiment 1

### Detection of Tobacco Mosaic Virus (TMV) in a Plant Sample

The TMV leads to the economically important mosaic disease of tobacco, but it can also infest other plant families. The virus is particularly stable and can be easily transmitted, e.g. by direct contact between the plants, by plant sap, in some plants by seed and most of all by agricultural methods for handling infected plants.

To avoid more serious economic damage an exact and quick identification of the virus is necessary.

For doing this, either pressed plant juice or plant parts (leaves, buds, fruit, trunks, stalks, roots, or similar parts) are used. If plant parts are used, they are lysed mechanically or chemically in a suitable buffer in the first step to release the viruses from the cell structure. Then, the obtained liquid is guided (like the pressed plant juice) through a filter array with decreasing pore size. Afterwards only the filters that catch the virus particles with a size from 15 nm to 400 nm are checked for the presence of viruses by applying the arrangement and method described before. The advantage of this procedure is the fact that other plant pathogenic viruses can be identified simultaneously.

### Embodiment 2

### Detection of the Foot-and-Mouth-Disease Virus

Foot-and-mouth disease is a highly contagious and compulsorily notifiable disease of cattle and pigs; but goats, sheep and other even-toed ungulates can also be infected. Infections of elephants, hedgehogs, rats and of men are described in the literature, too.

For example, aphtha liquid, organ homogenates, pharynx mucus samples (probang sample), secretions and cell culture supernatants can be used for identifying the virus. They are transferred to a suitable lysis buffer which leads to release of the viruses from the cells. Afterwards, the liquid obtained in this way is guided through the filter array mentioned in embodiment 1 and the filters of interest are analyzed as described.

### Embodiment 3

### Detection of Influenza Viruses in Air Samples

In humans, influenza is caused by the influenza virus of type A or B. The infection is often a result of a so called droplet or smear infection. The droplet infection is the medical term for the direct inhalation of expiration droplets (exhalation droplets) of infected persons.

Contact infection or smear infection with the viruses is caused by highly infectious expiration droplets that have fallen on objects or body surfaces or it is caused, for example, by smeared nasal secretion.

To identify viruses in an air sample a pre-defined volume of air is filtered in the already described filter array (cf. embodiment 1). Then, the filters are analyzed by means of the method explained above.

### Embodiment 4

### Detection of Bacteriophages in a Bacteria Culture

Viruses that use prokaryotes as host cells are generally called bacteriophages. The quick identification of bacteriophages is of particular interest here. They only attack bacteria and cause considerable damage in the bio-technological production of agents based on bacteria. The sample to be analyzed (culture medium, bacteria culture, or something similar) is first transferred to a suitable lysis buffer to break up the structures of the bacteria cells and to release the viruses. Afterwards, the solution is led through the filter array mentioned before and the filters are analyzed and evaluated as described.

1. Method for identifying individual viruses in a sample, comprising fixing the viruses from the sample on a carrier surface, scanning a height profile of the carrier surface with a probe, exposing at least selected scanning sites that are determined from the height profile of the carrier surface to monochromatic excitation light, registering in or at the probe a spectrum of Raman scattered light generated by the excitation light at each of the scanning sites exposed to the excitation light, and comparing said Raman scattered light registered at each of said scanning sites with reference values to identify an individual virus present at the corresponding scanning site.

2. Method according to claim 1, wherein the height profile scanning of the carrier surface with the determination of the selected scanning sites and the Raman scattered light registering in or at the probe for each of the selected scanning surface sites with the comparison with the reference values for the identifying of the viruses present at the corresponding scanning site are effected simultaneously.

3. Method according to claim 1, wherein the height profile scanning of the carrier surface with the determination of the selected scanning sites is effected before the exposing of each of the selected sites to monochromatic excitation light and the registering of the Raman scattered light for identifying the viruses present at the corresponding scanning site.

4. Method according to claim 1, wherein the reference values comprise data of an electronic database for identifying viruses.

5-8. (canceled)

9. Method according to claim 1, further comprising pre-treating the sample by filtration to effect sizing of the sample thereby to obtain a pre-treated sample in which concentration of viruses is greater than in the original sample.

10. Method according to claim 9, in which the sample comprises a liquid.

11. Method according to claim 9, in which the sample comprises a gas.

12. Method according to claim 11, in which the pre-treating further comprises dissolving the gas in a liquid and the filtration is of the liquid.

13. Method according to claim 1, in which the sample comprises a solid and the method further comprises pre-treating the sample by homogenization to effect sizing of the sample thereby to obtain a pre-treated sample in which concentration of viruses is greater than in the original sample.

14. Method according to claim 12, in which the homogenization comprises mechanical comminution.
